(19) United States
(12) Patent Application Publication
(10) Pub. No.: US 2022/0002754 A1
(43) Pub. Date: Jan. 6, 2022

(54) USE OF NW_006883358.1 IN CHO CELL GENOME FOR STABLE EXPRESSION OF PROTEIN

(71) Applicant: JIANGNAN UNIVERSITY, Wuxi (CN)

(72) Inventors: Yun CHEN, Wuxi (CN); Jian JIN, Wuxi (CN); Huazhong LI, Wuxi (CN); Songtao ZHOU, Wuxi (CN); Zuoying DUAN, Wuxi (CN); Xiaohai GONG, Wuxi (CN)

(21) Appl. No.: 17/289,015

(22) PCT Filed: Oct. 22, 2019
(86) PCT No.: PCT/CN2019/112449

§ 371 Date: Apr. 27, 2021

(51) Int. Cl. C12N 15/90; C12N 15/85

(52) U.S. Cl. CPC C12N 15/907; C12N 2510/02; C12N 15/85

(57) ABSTRACT

The invention provides use of NW_006883358.1 in a CHO cell genome for stable expression of a protein. A site for stable expression of a protein in the CHO cell genome is located at the 6874389^{th} base of CHO cell gene NW_006883358.1; 5′NNNNNNNNNNNNNNNNNNNNNGG3′ within a range of 6874325-6874443 near the site, which can be identified by CRISPR/Cas9 technology, is a target sequence. In the present application, different protein genes are introduced to a fixed position in the CHO cell genome for stable expression.

### FIELD OF THE INVENTION

The present invention relates to the field of genetic technology, and more particularly relates to a recombinant gene of CHO cells for stable expression of proteins.

### DESCRIPTION OF THE RELATED ART

Chinese Hamster Ovary (CHO) cells, as the main cell line in the field of bio-pharmaceuticals, has developed many different types of CHO cell lines, including cell lines that can be used to expand the number of gene copies. However, the increase of transgenic copy number is not positively correlated with the enhancement of target protein yield. Moreover, even with increased protein expression, the expression levels of most CHO cells are unstable. The current methods commonly used for constructing stable transfected cells are time and labor consuming, mainly due to that a large number of monoclonal screening processes need to be repeated. Therefore, it is widely expected currently in the field of cell line construction to develop a method capable of obtaining highly and stably expressed cells within a short time, meanwhile the constructed recombinant cell lines should be ensured to have the same qualities as those prepared from traditional processes to ensure the approval of regulators.

A traditional process for constructing foreign protein-expressing cell lines is to obtain cell lines highly expressing foreign proteins by randomly integrating foreign genes onto a cell genome and then conducting a series of screening on the highly expressed monoclonal cells. Due to the diversity of site effect differences, the expression levels of recombinant cells produced by random integration are different, so it will take a long time and many steps to select highly expressed monoclonal cells in the later stage. Monoclonal cells obtained through random integration cannot guarantee the stable expression of polypeptides/proteins during cell passage, and repeated monoclonal screenings are required for each recombinant cell construction, which increases the costs on research and development of biopharmaceuticals. Site effect hinders the efficiency of traditional random integration in the construction of recombinant cell lines, and repeated highly expressing monoclonal screenings are time and labor consuming and expensive. How to overcome the site effect and obtain stably expressed monoclonal cells rapidly and efficiently by using a site-specific integration technology has been discussed in academia for years, but no breakthrough has been made.

### SUMMARY OF THE INVENTION

To overcome the above problems in the prior art, the invention provides use of NW_006883358.1 in a CHO cell genome for stable expression of protein. According to the present invention, different protein genes are introduced to a fixed position in the CHO cell genome for stable expression; in addition, in the process of the site-specific integration, there is no need for multiple monoclonal screenings to obtain higher-expressing cell lines, thereby saving a lot of time.

For the above purpose, the invention utilizes the following technical solutions.

The invention provides use of a site within a CHO cell genome for stable expression of protein, the site for stable expression of protein within the CHO cell genome is located at the 6874389^{th} base of CHO cell gene NW_006883358.1; 5′ NNNNNNNNNNNNNNNNNNNNNGG3′ within a base range of 6874325-6874443 near the site, which can be identified by CRISPR/Cas9 technology, is a target sequence.

The protein has a molecular weight of less than 160 KDa.

The protein is selected from a polypeptide, a functional protein, an antibody, a fusion protein and any combination thereof.

The target sequence is within a base range of 6874379-6874401 near the 6874389^{th} base of the CHO cell gene NW_006883358.1.

Preferably, the target sequence is
5′-TCTTGCAGCCCTCTCCATTTTGG-3′.
Preferably, the target sequence is
5′-GCGGGGTCAGGCCCCAGTGCCGG-3′.
Preferably, the target sequence is
5′-TCAGGCCCCAGTGCCGGAAGTGG-3′.
Preferably, the target sequence is
5′-CCCAGTGCCGGAAGTGGAGTTGG-3′.
Preferably, the target sequence is
5′-GCAGCCCTCTCCATTTTGGCTGG-3′.
Preferably, the target sequence is
5′-CTCTCCATTTTGGCTGGGCTTGG-3′.
Preferably, the target sequence is
5′-ATTTTGGCTGGGCTTGGCCCTGG-3′.
Preferably, the target sequence is
5′-TTTTGGCTGGGCTTGGCCCTGGG-3′.
Preferably, the target sequence is
5′-TGGCTGGGCTTGGCCCTGGGTGG-3′.
Preferably, the target sequence is
5′-TGGATGTGAAGCCCGTCTCTCGG-3′.
Preferably, the target sequence is
5′-GGATGTGAAGCCCGTCTCTCGGG-3′.
Preferably, the target sequence is
5′-GATGTGAAGCCCGTCTCTCGGGG-3′.
Preferably, the target sequence is
5′-CGTCTCTCGGGGACATTTGTTGG-3′.
Preferably, the target sequence is
5′-GTCTCTCGGGGACATTTGTTGGG-3′.
Preferably, the target sequence is
5′-GACGGGCTTCACATCCACCCAGG-3′.
Preferably, the target sequence is
5′-ACGGGCTTCACATCCACCCAGGG-3′.
Preferably, the target sequence is
5′-AGGGCCAAGCCCAGCCAAAATGG-3′.
Preferably, the target sequence is
5′-AGGGCTGCAAGACAGCTTCGAGG-3′.
Preferably, the target sequence is
5′-AGTATGACCAACTCCACTTCCGG-3′.
Preferably, the target sequence is
5′-ACCAACTCCACTTCCGGCACTGG-3′.
Preferably, the target sequence is
5′-CCAACTCCACTTCCGGCACTGGG-3′.
Preferably, the target sequence is
5′-CAACTCCACTTCCGGCACTGGGG-3′.
Preferably, the target sequence is
5′-CACTGGGGCCTGACCCCGCCAGG-3′.
Preferably, the target sequence is
5′-ACTGGGGCCTGACCCCGCCAGGG-3′.
Preferably, the target sequence is
5′-CAACAAATGTCCCCGAGAGACGG-3′.
Preferably, the target sequence is
5′-CAGCCCTCTCCATTTTGGCTGGG-3′.
Preferably, the target sequence is
5′-CAAGCCCAGCCAAAATGGAGAGG-3′.
Preferably, the target sequence is
5′-AAGCCCAGCCAAAATGGAGAGGG-3′.

The invention also provides a targeting vector, and the targeting vector comprises the target sequence of 5′NNNNNNNNNNNNNNNNNNNNNGG3′ which can be used for site editing and can be identified by CRISPR/Cas9 technology.

Preferably, the targeting vector is prepared by digesting the PSK-u6-gRNA plasmid with BbsI and ligating it with a target sequence DNA fragment containing BbsI cohesive ends to obtain a targeting vector.

The invention also provides a recombinant donor vector for expressing a foreign gene in CHO cells. The recombinant donor vector comprises a homologous arm within a range of 500 bp-800 bp upstream and downstream of the 6874389^{th} base of the site NW_006883358.1.

Preferably, the recombinant donor vector is prepared by inserting a protein gene into a region between 5′ arm and 3′ arm of a plasmid, so that a nucleotide sequence is located downstream of a promoter and is regulated by the promoter, to get a recombinant CHO cell-expressed plasmid.

The promoter is selected from the group consisting of CMV (Human cytomegalovirus-derived strong mammalian expression promoter), EF-1a (Human elongation factor 1α-derived strong mammalian expression promoter), SV40 (Simian vacuolating virus 40-derived mammalian expression promoter), PGK1 (Phosphoglycerate kinase gene-derived mammalian promoter), UBC (Human ubiquitin C gene-derived mammalian promoter), human beta actin (0-actin gene-derived mammalian promoter), CAG (strong hybrid mammalian promoter).

The invention further provides a CHO recombinant cell line for stably expressing a foreign protein, and the vector and plasmid is used in the construction of recombinant cell line.

The invention still further provides a method of expressing proteins with CHO cell genes, comprising the following steps:

(1) CHO cells are co-transformed with Cas9, the targeting vector and the recombinant donor vector, to obtain recombinant CHO cells;

(2) the recombinant CHO cells are cultured on a plate, the supernatant is collected to detect the expression level, and the adherent recombinant CHO cells are suspension domesticated;

(3) The suspension domesticated recombinant CHO cells are cultured in a shaking flask, and the expression level of proteins is detected.

The present invention further provides a method for selecting a stable expression site in CHO cell genome:

1) Lentiviruses are constructed with fluorescent tags and their titers are deduced. The igk-luc genes are integrated into multiple cloning sites of the pLVX-CMV-MCS-T2A-Zsgreen vector, and then transfected into the HEK-293T cell together with the two plasmids pSPAX2 and pMD2G. The cell supernatants are harvested twice at 48 hour and 72 hour respectively, and collected for ultracentrifugation to get the lentivirus.

2) CHO cells are coated on a 6-well plate and cultured overnight. On the next day, the lentiviruses are diluted and CHO cells are infected at low MOI (MOI<1) (virus particles corresponding to each cell). After infection for 96 hours, the CHO cells are sorted by a flow cytometer, and the cells with the brightest fluorescence intensity are directly inoculated into a 96-well plate. One week later, when the cells have grown into monoclonal colonies, they are observed under a fluorescence microscope, and the brightest cells colony with normal morphology and growth are selected and transferred into a 24-well plate for expanding the cultivation. When the cells are cultured to a convergence degree near 90%, they are transferred into a 6-well plate for cultivation, and finally expanded into a culture dish of 10 cm for cultivation. A part of cells are cryopreserved and the remaining cells are cultured in large scale continually.

3) All gene integration sites of lentivirus in CHO cells are searched by a genome walking technology Lenti-X Integration Site Analysis Kit (Clontech: 631263).

Several cell lines with the brightest fluorescence intensity and normal morphology and growth rate are used as the materials, and genomic DNA is digested with three restriction endonucleases ADraI, SspI, and HpaI overnight. Wherein, a 100 L reaction system is formulated with 2.5 μg of genomic DNA and 80 U of restriction endonucleases, and digested at 37° C. overnight (16-18 hours).

The digested products are purified and recycled with a DNA Purification Kit. The digested genomic DNA 4.8 μL, together with 1.9 μL Genome Walker Adaptor (25 μM) and 0.5 μL T4 ligase, are formulated into a system of 8 μL for ligation at 16° C. overnight. The ligation system is heated at 70° C. for 5 minutes to inactivate the ligase. 32 μL TE buffer is added into each system to constitute a corresponding library.

The library is subjected to 2 rounds of nested PCR to amplify the LTR region and adjacent genomic regions. The related operating steps of PCR reaction can be conducted following the instruction of Lenti-X Integration Site Analysis Kit (Clontech: 631263).

Finally, the PCR products are subjected to electrophoresis. The main strips are cut and recovered, and then sequenced. After obtaining all lentivirus integration information for each cell line, the related information of CHO cell lines only containing single copy lentivirus integration is selected, and their sequence information is compared with CHO-K1 genomic information on BLAST to find highly expressed integration sites.

Due to the data update, through sequence alignment, the original site information at the 6874389^{th} base of NW_0068883358.1 in CHO cells is changed to the 1689^{th} base of NW_003626341.1 in CHO cells. The range of 6874325-6874443 is changed to 1635-1753.

As compared the prior art, the present invention has the following beneficial effects:

In the present invention, a site-specific integration method is employed to site-specifically integrating the target genes into a stable expression region, which can overcome the problem of integration site uncertainty caused by random integration, effectively avoid multiple rounds of highly expressing monoclonal screening, thereby effectively decreasing the research time for constructing a stably expressing cell line in bio-pharmaceuticals, and reducing the cost.

According to the present invention, protein genes are introduced to a fixed position in the CHO cell genome for stable expression.

### BRIEF DESCRIPTION OF THE DRAWINGS

FIG. 1 is a schematic diagram of the invention;

FIG. 2 shows the identification results of CHO cell gene with NGGH 75 KDa gene;

FIG. 3 shows the sequencing of OoPCR_fwd and OoPCR_rev on the CHO cell gene with NGGH 75 KDa gene;

FIG. 4 shows the expression of HSA in cells at different passages;

FIG. 5 shows the expression of NGGH in cells at different passages;

FIG. 6 shows the mass profile of antibody proteins secreted from each recombinant CHO cell every day.

### DETAILED DESCRIPTION OF THE PREFERRED EMBODIMENTS

The present invention will be further illustrated in combination with the accompanying figures and specific embodiments.

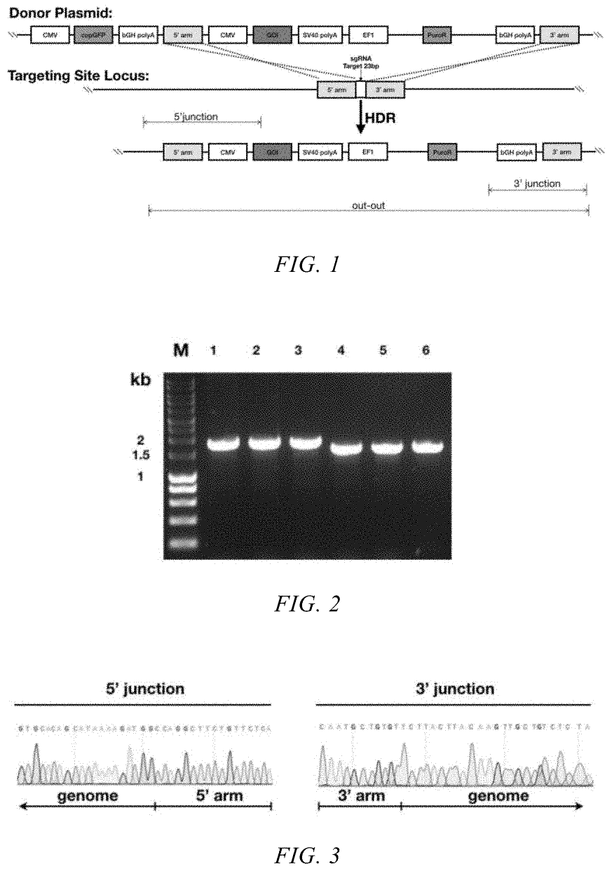

FIG. 1 shows a donor plasmid to be integrated to the site and how to site-specifically integrate the plasmid to the site through homologous recombination, wherein GOI is the target gene, which is site-specifically integrated to the target position through two homologous recombination arms, 5′arm and 3′arm, at a puromycin screening pressure of 4 μg/mL. In addition, the upstream sequence of 5′arm is a negative screening label, which can be used to remove monoclonal cells generated from random integration, thus ensuring that recombinant CHO cell lines generated from site-specific integration can be finally obtained.

### Embodiment 1

Selection of high expression sites;

Zsgreen1 gene is integrated at the 6874389(1689)^{th} base in NW_006883358.1(NW_003626341.1) (chromosome, 8). This fluorescent cell has been subcultured for no less than 50 generations. The expression level of fluorescence is detected with a flow cytometer. The expression level of green fluorescent protein is still good in the 50th generation of fluorescent cells, and the fluorescent signals can be retained stably during cell passages.

Additionally, this fluorescent cell is suspension domesticated, and the expression level of the suspension domesticated fluorescent protein is detected with a flow cytometer again. The detection results show that in recombinant CHO cells that have been suspended for 50 generations, more than 95% of the cells remain the expression of green fluorescent protein after suspension, which indicates that this site is extremely stable and will not lose the site-specifically integrated fluorescent protein genes due to cell passages.

### Embodiment 2

Selection of specific targets;

On the principle of proximity, the following sequence: 5′CCAACAAATGTCCCCGAGAGACGGGCTTCACATCCACCCAGGGCCAAGCC CAGCCAAAATGGAGAGGGCTGCAAGACAGCTTCGAGGAAAGAGTATGACC AACTCCACTTCCGGCACT 3′ is input to the CRISRPRater system to predict and sort out target sequences with low off-target efficiency. Wherein, the parameters are set as below: 1) The maximum number of mismatched bases at the beginning 15 bp after NGG is 0; 2) The number of mismatched bases at 21 bp after NGG is 2.

According to the above operation, the following sequence with a score of 0.79 is selected as the target sequence according to its scores:

5′-GCGGGGTCAGGCCCCAGTGCCGG-3′;

According to the CRISPRater system, LOW efficacy (score<0.56); MEDIUM efficacy (0.56<=score<=0.74); HIGH efficacy (score>0.74).

According to the CRISPRater evaluation system, all the target sequences within the range of 6874325-6874443 (1635-1753) near the site NW_006880285.1(NW_003626341.1) have obtained scores of greater than 0.56, which are in the range of moderate efficiency or high efficiency, and can be used as the 5′ NNNNNNNNNNNNNNNNNNNNNGG3′ target sequence identified by the CRISPR/Cas9 technology.

### Embodiment 3 Selection of Promoters

The above CMV (human cytomegalovirus-derived strong mammalian expression) promoter is replaced with different promoters, including common promoters such as EF-1a (human elongation factor 1α-derived strong mammalian expression promoter), SV40 (Simian vaculating virus 40-derived mammalian expression promoter), PGK1 (phosphoglycerate kinase gene-derived mammalian promoter), UBC (human ubiquitin C gene-derived mammalian promoter), human beta actin (β-actin gene-derived mammalian promoter) or CAG (strong hybrid mammalian promoter). Upon testing, all the above promoters can regulate the gene sequences of the downstream red fluorescent proteins and express the corresponding red fluorescent proteins.

### Embodiment 4

The human serumalbumin genes (HSA, 68 KDa) are site-specifically integrated at specific sites: To construct CRISPR/Cas9-mediated homologous recombination at a later stage, sgRNA and Donor Plasmid need to be constructed, for which the method is as below:

1. Construction of SgRNA, the Following Sequences are Firstly Synthesized:

sgRNA-1fwd
5′ TTTGGCGGGGTCAGGCCCCAGTGCGT3′
sgRNA-1rev
5′TAAAACGCACTGGGGCCTGACCCCGC3′

1) The PSK-u6-gRNA plasmid is digested with BBsI, and the digested vector is recovered;

2) The synthesized fragment is annealed to a double strand containing cohesive ends

sgRNA-1fwd (100 μM) 4 μL

sgRNA-1rev (100 μM) 4 μL

10×NEB buffer 22 μL

H₂O 10 μL

-------------------------------------

20 μL

water bath at 95° C. for 5 min, naturally cooling to room temperature in the water bath pot;

3) Fragment ligation and construction of the recombinant plasmid Recovered linear vector 50 ng

| | |
|---|---|
| Annealed fragment 1 μL | Ligation at room temperature |
| 10 × T4 ligase buffer 1 μL | for more than one hour, or |
| T4 ligase 1 μL | ligation at 4° C. overnight |
| H₂O | |
| To 10 μL; | |

4) Ligation and transformation;

5) Selection of clones, PCR identification, the primers used for identification are:

M13-synthesized R primers, those clones which can be amplified into bands are positive clones.

2. Construction of Donor Plasmid

The specific information of donor plasmid is as shown in FIG. 1: Except for GOI, the remaining parts are all synthesized; the sequence within 600 bp upstream and downstream of the target is the left and right homologous arm sequence information of the donor plasmid, and GOI is a process in which HSA genes are integrated onto the donor plasmid through the existing C115 kit from Vazyme Biotech Co.

3. Three plasmids, Cas9 (donated by Dr. Helene F Kildegaard, Technical University of Denmark), SgRNA and Donor plasmid are co-transfected with CHO cells which are cultured at 37° C. and 5% CO2, wherein the molar ratio of the three plasmids is 1:1:1, the transfection reagent is Lipofectamine 3000 (Thermo Fisher Scientific), and the specific transfection process refers to the instruction. After then, 4 μg/ml puromycin is added into the cells for screening, the process lasts for 10 days totally; monoclonal cells are sorted by a MoFloXDP FACS machine (Beckman Coulter), and cells without any fluorescence are selected and inoculated into a 96-well plate.

4. After growing for 2 weeks, a part of cells are identified by PCR, such as 5′junction PCR, 3′Junction PCR and out-out PCR. Positive cells are reserved.

### Embodiment 5

The glucagon-like peptide-1-human serum albumin fusion protein genes (NGGH, 75 KDa) are site-specifically integrated at specific sites: To construct CRISPR/Cas9-mediated homologous recombination at a later stage, sgRNA and Donor Plasmid need to be constructed, for which the method is as below: 1. The construction of sgRNA is the same as in Embodiment 4.

2. Construction of Donor plasmid.

The specific information of donor plasmid is as shown in FIG. 1: Except for GOI, the remaining parts are all synthesized; the sequence within 600 bp upstream and downstream of the target is the left and right homologous arm sequence information of the donor plasmid, and GOI is a process in which NGGH genes are integrated onto the donor plasmid through the existing C115 kit from Vazyme Biotech Co.

3. Three plasmids, Cas9, SgRNA and Donor plasmid are co-transfected with CHO cells which are cultured at 37° C. and 5% CO2, wherein the molar ratio of the three plasmids is 1:1:1, the transfection reagent is Lipofectamine 3000 (Thermo Fisher Scientific), and the specific transfection process refers to the instruction. After then, 4 μg/ml puromycin is added into the cells for screening, the process lasts for 10 days totally. Monoclonal cells are sorted by a MoFloXDP FACS machine (Beckman Coulter), and cells without any fluorescence are selected and inoculated into a 96-well plate.

4. After growing for 2 weeks, a part of cells are identified by PCR, such as 5′junction PCR, 3′Junction PCR and out-out PCR. Positive cells are reserved.

FIG. 2 shows the identification results of CHO gene with NGGH 75 KDa gene; wherein lanes 1-3 show 5′junction PCR results of three monoclonal cells, and lanes 4-6 show 3′junction PCR results; there are obvious strips in all the lanes, indicating that there is gene knock-in.

FIG. 3 shows that OoPCR_fwd and OoPCR_rev are utilized for sequencing to determine the sequence accuracy at the junction (for 5′junction and 3′junction, the junction between the 5′ upstream and the genome), wherein the sequencing results confirm that GOI is accurately inserted into the target area.

### Embodiment 6

The antibody genes (Avastin, 160 KDa) are site-specifically integrated at specific sites: To construct CRISPR/Cas9-mediated homologous recombination at a later stage, sgRNA and Donor Plasmid need to be constructed, for which the method is as below:

1. The construction of sgRNA is the same as in Embodiment 4.

2. Construction of Donor plasmid.

The specific information of donor plasmid is as shown in FIG. 1: Except for GOI, the remaining parts are all synthesized; the sequence within 600 bp upstream and downstream of the target is the left and right homologous arm sequence information of the donor plasmid, and GOI is a process in which Avastin genes are integrated onto the donor plasmid through the existing C115 kit from Vazyme Biotech Co.

3. Three plasmids, Cas9 (donated by Dr. Helene F Kildegaard, Technical University of Denmark), SgRNA and Donor plasmid are co-transfected with CHO cells which are cultured at 37° C. and 5% CO2, wherein the molar ratio of the three plasmids is 1:1:1, the transfection reagent is Lipofectamine 3000 (Thermo Fisher Scientific), and the specific transfection process refers to the instruction. After then, 4 μg/mL puromycin is added into the cells for screening, the process lasts for 10 days totally. Monoclonal cells are sorted by a MoFloXDP FACS machine (Beckman Coulter), and cells without any fluorescence are selected and inoculated into a 96-well plate.

4. After growing for 2 weeks, a part of cells are identified by PCR, such as 5′junction PCR, 3′Junction PCR and out-out PCR. Positive cells are reserved.

### Test Embodiment

The three cell lines obtained in Embodiments 4-6 are detected through ELISA to observe whether there is a target protein expression and whether it is a stable and long-term expression.

Detection method: The three detections are all conducted by the ELISA method, and all the sorted positive cells are cultured in a 6-well plate to determine whether there is a long-term stable expression of the target protein. The kits used for the experiments are Human Albumin ELISA Kit (RK00157) and Human IgG (Total) ELISA Kit (RK00393) from Abclonal Co.

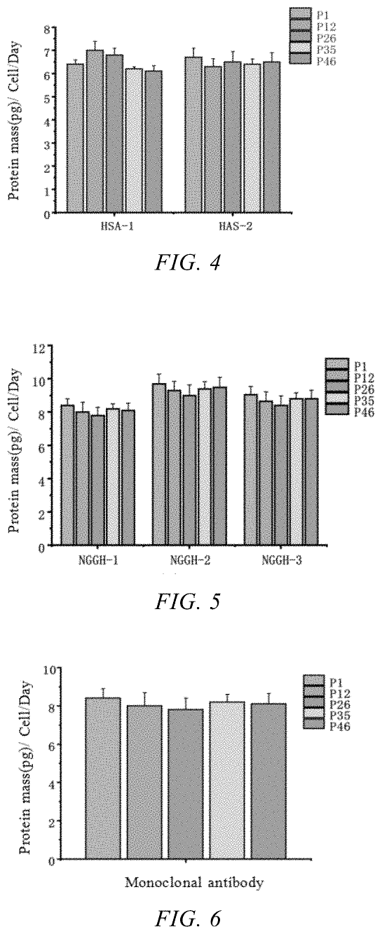

FIGS. 4 and 5 show the expression of HSA and NGGH at different passages respectively, wherein the vertical coordinate indicates the mass of proteins secreted from each cell every day.

It can be seen from the figures that both NGGH and HAS can stably express the corresponding genes in the plate within 50 passages, and the expression levels of the target protein by the sorted three NGGH site-specific integrated cell lines and two HAS site-specific integrated cell lines are close to each other.

FIG. 6 shows the mass of antibody proteins secreted from each recombinant CHO cell every day. Obviously, cells can stably express the secreted corresponding proteins at different passages, and this indicates good stabilities and is consistent with the previous results of fluorescent cells. The results show that this site can be subjected to CRISPR/Cas9-mediated site-specific integration, and can stably express the corresponding proteins.

After 5′-GCGGGGTCAGGCCCCAGTGCCGG-3′ sequence is selected to subject to the above tests, good results have been obtained. Therefore, the target sequences described in claims 5-31 all can be used to construct site-specifically integrated and stably expressing cell lines, all of which can stably express target proteins.

1. Use of a site within a CHO cell genome for stable expression of a protein, wherein the site for stable expression of a protein within the CHO cell genome is located at the 6874389^{th} base of CHO cell gene NW_006883358.1;
5′NNNNNNNNNNNNNNNNNNNNNGG3′ within a base range of 6874325-6874443 near the site, which can be identified by CRISPR/Cas9 technology, is a target sequence.

2. The use according to claim 1, wherein the protein has a molecular weight of less than 160 KDa.

3. The use according to claim 1, wherein the protein is selected from a polypeptide, a functional protein, an antibody, a fusion protein and any combination thereof.

4. The use according to claim 1, wherein the target sequence is within a range of 6874379-6874401^{th} bases near the 6874389^{th} base of the CHO cell gene NW_006883358.1; and the target sequence is 5′-TCTTGCAGCCCTCTCCATTTTGG-3′.

5. The use according to claim 1, wherein the target sequence is a sequence selected from the group consisting of 5′-GCGGGGTCAGGCCCCAGTGCCGG-3′, 5′-TCAGGCCCCAGTGCCGGAAGTGG-3′, 5′-CCCAGTGCCGGAAGTGGAGTTGG-3′, 5′-GCAGCCCTCTCCATTTTGGCTGG-3′, 5′-CTCTCCATTTTGGCTGGGCTTGG-3′, 5′-ATTTTGGCTGGGCTTGGCCCTGG-3′, 5′-TTTTGGCTGGGCTTGGCCCTGGG-3′, 5′-TGGCTGGGCTTGGCCCTGGGTGG-3′, 5′-TGGATGTGAAGCCCGTCTCTCGG-3′, 5′-GGATGTGAAGCCCGTCTCTCGGG-3′, 5′-GATGTGAAGCCCGTCTCTCGGGG-3′, 5′-CGTCTCTCGGGGACATTTGTTGG-3′, 5′-GTCTCTCGGGGACATTTGTTGGG-3′, 5′-GACGGGCTTCACATCCACCCAGG-3′, 5′-ACGGGCTTCACATCCACCCAGGG-3′, 5′-AGGGCCAAGCCCAGCCAAAATGG-3′, 5′-AGGGCTGCAAGACAGCTTCGAGG-3′, 5′-AGTATGACCAACTCCACTTCCGG-3′, 5′-ACCAACTCCACTTCCGGCACTGG-3′, 5′-CCAACTCCACTTCCGGCACTGGG-3′, 5′-CAACTCCACTTCCGGCACTGGGG-3′, 5′-CACTGGGGCCTGACCCCGCCAGG-3′, 5′-ACTGGGGCCTGACCCCGCCAGGG-3′, 5′-CAACAAATGTCCCCGAGAGACGG-3′, 5′-CAGCCCTCTCCATTTTGGCTGGG-3′, 5′-CAAGCCCAGCCAAAATGGAGAGG-3′, and 5′-AAGCCCAGCCAAAATGGAGAGGG-3′.

6-31. (canceled)

32. A recombinant donor vector for expressing a foreign gene in CHO cells, wherein the recombinant donor vector comprises a homologous arm within a range of 600 bp upstream and downstream of the 6874389^{th} base of the site NW_006883358.1 according to claim 1.

33. The recombinant donor vector according to claim 32, wherein the vector is prepared by inserting a protein gene into a region between 5′ arm and 3′ arm of a plasmid, so that a nucleotide sequence is located downstream of a promoter and is regulated by the promoter, to get a recombinant CHO cell-expressed plasmid.

34. A CHO recombinant cell line for stably expressing a foreign protein, wherein the vector or plasmid according to claim 32 is used in the construction of the recombinant cell line.


SEQUENCE LISTING (31 sequences)

SEQ ID NO: 1 - DNA, 23 bp, Artificial sequence
  target sequence
tcttgcagccctctccattttgg

SEQ ID NO: 2 - DNA, 23 bp, Artificial sequence
  target sequence
gcggggtcaggccccagtgccgg

SEQ ID NO: 3 - DNA, 23 bp, Artificial sequence
  target sequence
tcaggccccagtgccggaagtgg

SEQ ID NO: 4 - DNA, 23 bp, Artificial sequence
  target sequence
cccagtgccggaagtggagttgg

SEQ ID NO: 5 - DNA, 23 bp, Artificial sequence
  target sequence
gcagccctctccattttggctgg

SEQ ID NO: 6 - DNA, 23 bp, Artificial sequence
  target sequence
ctctccattttggctgggcttgg

SEQ ID NO: 7 - DNA, 23 bp, Artificial sequence
  target sequence
attttggctgggcttggccctgg

SEQ ID NO: 8 - DNA, 23 bp, Artificial sequence
  target sequence
ttttggctgggcttggccctggg

SEQ ID NO: 9 - DNA, 23 bp, Artificial sequence
  target sequence
tggctgggcttggccctgggtgg

SEQ ID NO: 10 - DNA, 23 bp, Artificial sequence
  target sequence
tggatgtgaagcccgtctctcgg

SEQ ID NO: 11 - DNA, 23 bp, Artificial sequence
  target sequence
ggatgtgaagcccgtctctcggg

SEQ ID NO: 12 - DNA, 23 bp, Artificial sequence
  target sequence
gatgtgaagcccgtctctcgggg

SEQ ID NO: 13 - DNA, 23 bp, Artificial sequence
  target sequence
cgtctctcggggacatttgttgg

SEQ ID NO: 14 - DNA, 23 bp, Artificial sequence
  target sequence
gtctctcggggacatttgttggg

SEQ ID NO: 15 - DNA, 23 bp, Artificial sequence
  target sequence
gacgggcttcacatccacccagg

SEQ ID NO: 16 - DNA, 23 bp, Artificial sequence
  target sequence
acgggcttcacatccacccaggg

SEQ ID NO: 17 - DNA, 23 bp, Artificial sequence
  target sequence
agggccaagcccagccaaaatgg

SEQ ID NO: 18 - DNA, 23 bp, Artificial sequence
  target sequence
agggctgcaagacagcttcgagg

SEQ ID NO: 19 - DNA, 23 bp, Artificial sequence
  target sequence
agtatgaccaactccacttccgg

SEQ ID NO: 20 - DNA, 23 bp, Artificial sequence
  target sequence
accaactccacttccggcactgg

SEQ ID NO: 21 - DNA, 23 bp, Artificial sequence
  target sequence
ccaactccacttccggcactggg

SEQ ID NO: 22 - DNA, 23 bp, Artificial sequence
  target sequence
caactccacttccggcactgggg

SEQ ID NO: 23 - DNA, 23 bp, Artificial sequence
  target sequence
cactggggcctgaccccgccagg

SEQ ID NO: 24 - DNA, 23 bp, Artificial sequence
  target sequence
actggggcctgaccccgccaggg

SEQ ID NO: 25 - DNA, 23 bp, Artificial sequence
  target sequence
caacaaatgtccccgagagacgg

SEQ ID NO: 26 - DNA, 23 bp, Artificial sequence
  target sequence
cagccctctccattttggctggg

SEQ ID NO: 27 - DNA, 23 bp, Artificial sequence
  target sequence
caagcccagccaaaatggagagg

SEQ ID NO: 28 - DNA, 23 bp, Artificial sequence
  target sequence
aagcccagccaaaatggagaggg

SEQ ID NO: 29 - DNA, 26 bp, Artificial sequence
  sgRNA-1fwd
tttggcggggtcaggccccagtgcgt

SEQ ID NO: 30 - DNA, 26 bp, Artificial sequence
  sgRNA-1rev
taaaacgcactggggcctgaccccgc

SEQ ID NO: 31 - DNA, 119 bp, Artificial sequence
  target sequence
cccaacaaatgtccccgagagacgggcttcacatccacccagggccaagcccagccaaaatggagagggctgcaagacagcttcgaggaaagagtatgaccaactccacttccggcact